(19) United States
(12) Patent Application Publication
(10) Pub. No.: US 2024/0016832 A1
(43) Pub. Date: Jan. 18, 2024

(54) DIALYSATE FOR HEMODIALYSIS

(71) Applicants: Masaaki NAKAYAMA, Tokyo (JP); Yoshiki NISHIZAWA, Osaka (JP); RIKEN, Saitama (JP); NIHON TRIM CO., LTD., Osaka (JP)

(72) Inventors: Masaaki NAKAYAMA, Tokyo (JP); Yasuyoshi WATANABE, Saitama (JP); Yoshiki NISHIZAWA, Osaka (JP); Shigeru KABAYAMA, Osaka (JP)

(21) Appl. No.: 18/352,132

(22) Filed: Jul. 13, 2023

(30) Foreign Application Priority Data
Jul. 13, 2022 (JP) 2022-112133

(51) Int. Cl. A61K 33/00; A61P 13/12

(52) U.S. Cl. CPC A61K 33/00; A61P 13/12

(57) ABSTRACT

A dialysate for hemodialysis contains electrolyzed hydrogen water, has a dissolved hydrogen concentration of 100 ppb to 200 ppb, and has an activity of reducing a feeling of fatigue.

### CROSS-REFERENCE TO RELATED APPLICATION

This application claims priority to Japanese Patent Application No. 2022-112133 filed on Jul. 13, 2022, the entire disclosure of which is incorporated by reference herein.

### BACKGROUND

The present disclosure relates to dialysates for hemodialysis.

Hemodialysis is known as one of effective treatments for renal failure patients who are unable to urinate to control the water level in the body and remove toxic substances including wastes such as urea from the body due to their kidneys not functioning well enough.

Hemodialysis is a procedure in which the following operation is continuously performed. Blood is drawn out of the body by a blood pump and is brought into contact with a dialysate through a dialyzer to remove toxic substances and excess water from the blood using a diffusion phenomenon caused by a concentration gradient, and the blood is then returned into the body (blood retransfusion).

Regarding the dialysate, a method for producing a dialysate containing dissolved hydrogen has been proposed (see, for example, Japanese Unexamined Patent Publication No. 2016-13349). In this method, for example, a dialysate is produced by bubbling hydrogen gas through water purified by a reverse osmosis membrane (RO membrane) (hereinafter referred to as “reverse osmosis water”) so that the reverse osmosis water contains dissolved hydrogen.

In recent years, it has been known that patients on hemodialysis using the conventional dialysate suffer from oxidative stress. This is considered to be due to active oxygen generated during dialysis. Oxidative stress is a significant factor that impairs the patients' quality of life (QOL) because it causes complications such as a feeling of fatigue during and immediately after the treatment. In particular, a feeling of severe fatigue that not only is felt on the day of hemodialysis but also lasts until the day after hemodialysis is a factor that adversely affects life prognosis and hinders reintegration into society.

However, it is difficult to eliminate active oxygen by the dialysate described in Japanese Unexamined Patent Publication No. 2016-13349, and it is therefore not possible to reduce the patients' feeling of fatigue from oxidative stress by this dialysate.

The present invention was made in view of the above problem, and it is an object of the present invention to provide a dialysate that can dramatically reduce the feeling of fatigue of patients with severe fatigue in a short period of time by eliminating active oxygen and thus reducing oxidative stress.

### SUMMARY

In order to achieve the above object, a dialysate for hemodialysis according to the present invention contains electrolyzed hydrogen water, has a dissolved hydrogen concentration of 100 ppb to 200 ppb, and has an activity of reducing a feeling of fatigue.

The dialysate according to the present invention has an enhanced antioxidative effect on dialysis patients, and can therefore dramatically reduce the feeling of fatigue of patients with severe fatigue from oxidative stress in a short period of time.

### BRIEF DESCRIPTION OF THE DRAWINGS

FIG. 1 is a conceptual diagram showing the configuration of an apparatus for producing a dialysate for hemodialysis according to an embodiment of the present invention.

FIG. 2 shows an electrolytic cell in an electrolysis module of the apparatus for producing a dialysate for hemodialysis according to the embodiment of the present invention.

FIG. 3 shows a dialysis machine according to the embodiment of the present invention.

FIG. 4 shows the results of a Visual Analogue Scale (VAS) for the severity of a feeling of fatigue in examples.

### DETAILED DESCRIPTION

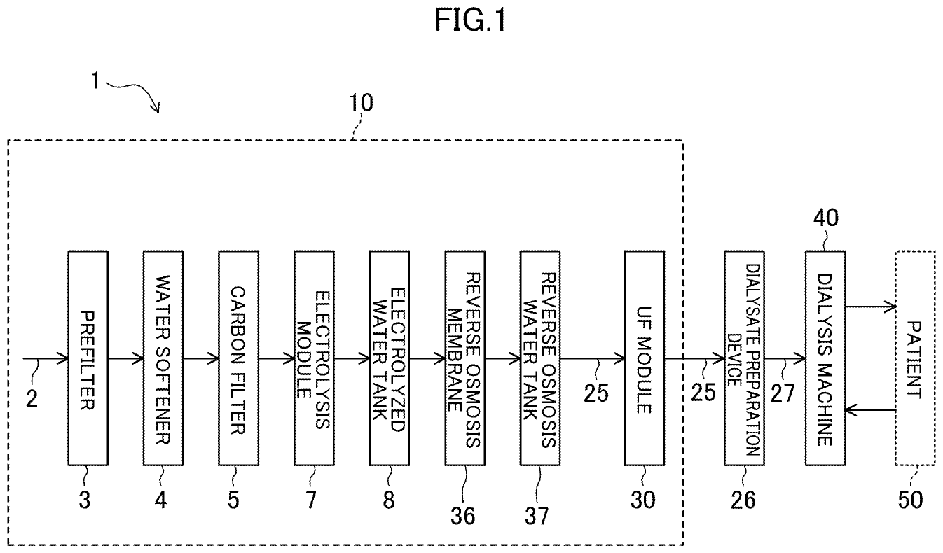

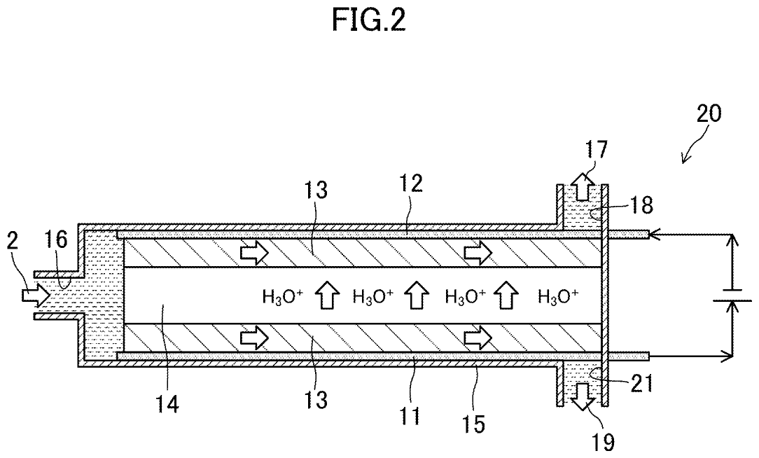

FIG. 1 is a conceptual diagram showing the configuration of an apparatus for producing a dialysate for hemodialysis according to an embodiment of the present invention. FIG. 2 shows an electrolytic cell in an electrolysis module of the apparatus for producing a dialysate for hemodialysis according to the embodiment of the present invention.

As used herein, the “dialysate for hemodialysis” can also be used as infusion solutions such as a replacement solution and a replenishment solution for other blood purification therapies such as hemofiltration and hemodiafiltration.

The components of the dialysate for hemodialysis include various electrolytes such as sodium ions (Na⁺), potassium ions (K⁺), calcium ions (Ca²⁺), magnesium ions (Mg²⁺), chloride ions (Cl⁻), acetate ions (CH₃COO⁻), and bicarbonate ions (HCO₃⁻) and components suitable as components of a dialysate for hemodialysis, such as glucose, lactic acid, citric acid, sugars, polysaccharides, and amino acid.

The “dialysate for hemodialysis” in the present invention will be hereinafter simply referred to as “dialysate.”

As shown in FIG. 1, a dialysate production apparatus 1 includes a dialysis water production device 10 and a dialysate preparation device 26.

The dialysis water production device 10 includes a prefilter 3, a water softener 4 connected to the prefilter 3, a carbon filter (activated carbon treatment device) 5 connected to the water softener 4, an electrolysis module 7 connected to the carbon filter 5, an electrolyzed water tank 8 connected to the electrolysis module 7, a reverse osmosis membrane 36 connected to the electrolyzed water tank 8, and an Ultra Filter (UF) module 30 connected to the reverse osmosis membrane 36.

The prefilter 3 is a filter that removes impurities (e.g., iron rust and sand particles) from raw water 2 (hard water containing dissolved solids such as calcium ions and magnesium ions that are hardness components).

The water softener 4 is a device that softens the raw water 2 by removing the hardness components from the raw water 2 through a substitution reaction by ion exchange. In the present embodiment, the raw water 2 can be, for example, tap water, well water, or groundwater.

The carbon filter 5 is a filter that removes residual chlorine, chloramine, organic matter, etc. from the raw water 2 treated by the water softener 4 through physical adsorption by using activated carbon that is a porous adsorbent.

The water softener 4 and the carbon filter 5 can be a known water softener and a known carbon filter.

The electrolysis module 7 is a module that functions as a hydrogen dissolving device and that electrolyzes the raw water 2 filtered through the carbon filter 5 to produce water containing dissolved hydrogen (dissolved hydrogen water) to be used as water for dialysate preparation.

The electrolysis module 7 of the present embodiment includes an electrolytic cell 20 with a solid polymer membrane (solid polymer electrolyte membrane) 14 as shown in FIG. 2.

As shown in FIG. 2, the electrolytic cell 20 includes the solid polymer membrane 14, an anode 11, a cathode 12, and dielectric layers 13. The anode 11 and the cathode 12 are conductors that supply current to the electrolytic cell 20, and are disposed so as to face each other with the solid polymer membrane 14 interposed therebetween. The dielectric layers 13 are disposed between the solid polymer membrane 14 and the anode 11 and between the solid polymer membrane 14 and the cathode 12.

As shown in FIG. 2, the anode 11 and the cathode 12 are electrically connected to each other, and the solid polymer membrane 14, the anode 11, the cathode 12, and the dielectric layers 13 are housed in an electrolytic cell body 15.

As shown in FIG. 2, the electrolytic cell body 15 has an inlet channel 16 for introducing the raw water 2 to be electrolyzed into the electrolytic cell body 15.

Examples of the material of the anode 11 and cathode 12 include titanium and platinum.

Examples of the material of the dielectric layers 13 include titanium and platinum.

The solid polymer membrane 14 serves to move oxonium ions (H₃O⁺) produced at the anode 11 toward the cathode 12 by electrolysis.

For example, the solid polymer membrane 14 is suitably a solid polymer membrane made of a fluorine resin material with sulfonic acid groups. Specific suitable examples of the solid polymer membrane 14 of the present invention include commercially available products such as Nafion (made by DuPont), Flemion (made by AGC Inc.), or Aciplex (made by AGC Inc.).

In the electrolysis that is performed in the electrolysis module 7 using such a solid polymer membrane 14, oxonium ions (H₃O⁺) are used as a raw material for hydrogen production at the cathode 12, and no OH⁻ ions are produced during the electrolysis process in the electrolysis module 7. Therefore, even if the electrolysis process is performed at high current in order to increase the amount of dissolved hydrogen, the pH of the treated water does not change.

This overcomes the disadvantage that the dissolved hydrogen concentration of the treated water is reduced due to the upper limit of the pH. As a result, the electrolysis process can be performed at desired high current and the dissolved hydrogen concentration of the treated water can be improved. It is therefore possible to obtain treated water with a required dissolved hydrogen concentration.

Treated water (electrolyzed hydrogen water) 17 produced by the above electrolysis process is sent to the electrolyzed water tank 8 connected to the electrolysis module 7 through a water supply channel 18 located on the cathode side of the electrolytic cell body 15. Water containing dissolved oxygen (dissolved oxygen water) 19 produced on the anode side by the electrolysis process is discharged to the outside through a drain channel 21 located on the anode side of the electrolytic cell body 15.

The electrolyzed water tank 8 is a tank that stores the electrolyzed hydrogen water produced by the electrolysis module 7.

There is a phenomenon (osmosis) in which water moves from a lower concentration solution to a high concentration solution when solutions on both sides of a semipermeable membrane have different concentrations from each other. The reverse osmosis membrane 36 is a membrane that uses high pressure to force water in a higher concentration solution to pass through the membrane into a lower concentration solution to produce reverse osmosis water (reverse osmosis membrane treatment). The electrolyzed hydrogen water produced by the electrolysis module 7 is subjected to this reverse osmosis membrane treatment using the reverse osmosis membrane 36.

Since the reverse osmosis membrane 36 can further remove impurities such as trace metals from the raw water obtained by the above series of processes, water meeting the requirements defined by ISO 13959 (water quality standard for dialysis water) can be obtained.

As shown in FIG. 1, the reverse osmosis membrane 36 is connected to a reverse osmosis water tank 37 for storing reverse osmosis water 25 obtained by the reverse osmosis membrane treatment.

The UF module 30 is connected to the reverse osmosis water tank 37. The UF module 30 is a module that removes bacteria and microorganisms from the reverse osmosis water 25.

As shown in FIG. 1, the dialysate preparation device 26 is connected to the UF module 30, and the reverse osmosis water 25 treated by the UF module 30 is supplied as water for dialysate preparation to the dialysate preparation device 26.

The dialysate preparation device 26 prepares a dialysate 27 by mixing the supplied reverse osmosis water 25 and a dialysis stock solution, and supplies the dialysate 27 to a dialysis machine 40 connected to the dialysate preparation device 26. The dialysate 27 thus supplied to the dialysis machine 40 is used to clean the blood of a patient 50. That is, the dialysate preparation device 26 also functions as a dialysate supply device that supplies the prepared dialysate 27 to the dialysis machine 40.

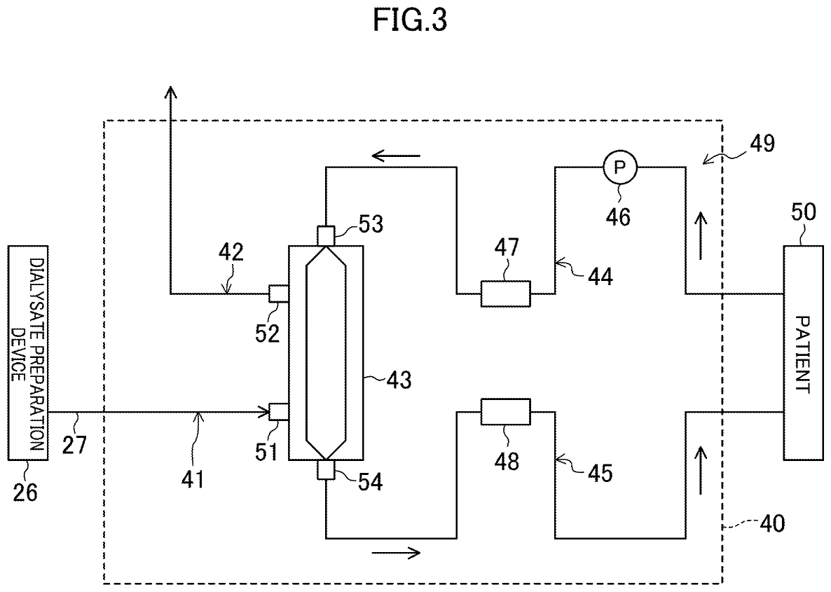

FIG. 3 shows the dialysis machine 40 according to the embodiment of the present invention. As shown in FIG. 3, the dialysis machine 40 includes a blood circuit 49 and a dialyzer 43. The blood circuit 49 is composed of an arterial blood circuit 44 and a venous blood circuit 45, and the dialyzer 43 is connected to the arterial blood circuit 44 and the venous blood circuit 45. The dialyzer 43 is a blood cleaning device that cleans blood flowing through the blood circuit 49.

The dialysis machine 40 further includes a dialysate inlet channel 41 and a dialysate discharge channel 42. The dialysate inlet channel 41 is connected to the dialysate preparation device 26 and the dialyzer 43 and introduces the dialysate 27 prepared by the dialysate preparation device 26 into the dialyzer 43. The dialysate discharge channel 42 is connected to the dialyzer 43, and discharges the dialysate 27 introduced into the dialyzer 43 together with wastes in the blood.

The dialysis machine 40 further includes a blood circulation pump 46 and defoamers 47, 48. The defoamer 47 is located in the arterial blood circuit 44, and the defoamer 48 is located in the venous blood circuit 45.

The dialyzer 43 is connected to the dialysate inlet channel 41, the dialysate discharge channel 42, the arterial blood circuit 44, and the venous blood circuit 45 via connectors (couplers) 51 to 54 shown in FIG. 3, respectively. The dialyzer 43 is detachably attached to these connectors 51 to 54.

In the present embodiment, when the pump 46 is driven with an arterial puncture needle and a venous puncture needle (both not shown) inserted into the patient 50, the blood of the patient 50 is defoamed by the defoamer 47, sent to the dialyzer 43 through the arterial blood circuit 44, and then cleaned by the dialyzer 43. If not defoamed by the defoamer 47, air bubbles may get into the blood and may cause embolism.

The dialysis machine 40 includes an air bubble detector (not shown). When an air bubble passes through the air bubble detector, it generates an air bubble alarm and stops operating (that is, stops the pump 46 and disconnects the blood circuit 49 from the outside to prevent air from entering the body). In this case, it is necessary to check and restart the dialysis machine 40, which makes it difficult to stably perform dialysis and increases the time required for dialysis.

The blood cleaned by the dialyzer 43 is then defoamed by the defoamer 48 and returned to the body of the patient 50 through the venous blood circuit 45.

The dialyzer 43 contains a plurality of hollow fibers with a predetermined inner diameter (e.g., 200 μm). The inside of each hollow fiber serves as a channel for the blood, and the space between the outer peripheral surface of each hollow fiber and the inner peripheral surface of a housing portion of the dialyzer 43 serves as a channel for the dialysate 27.

Each hollow fiber has a large number of very small pores extending through its outer and inner peripheral surfaces, so that a wall of each hollow fiber serves as a semipermeable membrane. Impurities etc. in the blood pass through the semipermeable membranes into the dialysate 27.

The inventors found that performing dialysis (electrolyzed water dialysis) using the dialysate 27 containing electrolyzed hydrogen water and having a predetermined dissolved hydrogen concentration eliminates active oxygen generated during the dialysis and thus reduces oxidative stress, and as a result, reduces patients' feeling of fatigue. The inventors completed the invention based on this finding.

More specifically, administering the dialysate 27 containing the electrolyzed hydrogen water according to the present invention to the patient 50 reduces blood myeloperoxidase and monocyte chemotactic factor (monocyte chemoattractant protein-1 (MCP-1)) and thus reduces in-vivo oxidative stress. The feeling of fatigue of the patient 50 with severe fatigue from oxidative stress can thus be dramatically reduced in a short period of time.

As a result, the clinical use of the dialysate of the present invention can improve the QOL of patients on hemodialysis, and can help them reintegrate into society and return to their home life.

As used herein, the “patient with severe fatigue” refers to a patient who has a feeling of fatigue not only on the day of dialysis but also on the following day, and the “feeling of fatigue of the patient with severe fatigue” refers to a persistent feeling of fatigue the patient who has a feeling of fatigue not only on the day of dialysis but also on the following day subjectively feels from hemodialysis.

The dissolved hydrogen concentration of the dialysate 27 is 100 ppm to 200 ppb. The dialysate 27 with a dissolved hydrogen concentration of less than 100 ppb may not sufficiently reduce the feeling of fatigue due to the low dissolved hydrogen concentration. The dialysate 27 with a dissolved hydrogen concentration of 200 ppb or less effectively reduces generation of air bubbles in the dialysate 27. This reduces the risk of thrombus formation etc. by air bubbles, so that dialysis can be safely performed. This also reduces the possibility of both a decrease in electrolysis efficiency and an air lock due to air bubbles sticking to channels of the dialysis water production device 10 etc., so that the dialysate can be stably provided.

### Examples

The present invention will be described below based on examples. The present invention is not limited to the examples. The examples can be modified and changed based on the spirit and scope of the present invention, and such modifications and changes are not excluded from the scope of the invention.

### Production of Dialysate

Dialysates were produced using the dialysis water production device 10 of FIG. 1 including the electrolysis module 7. More specifically, reverse osmosis water was produced using a dialysis water production device for multipatient electrolyzed water dialysis (made by TRIM MEDICAL INSTITUTE CO., LTD., product name: EW-SP754-HD) including the electrolysis module 7. The dialysates of the examples (dissolved hydrogen concentration of 120 ppb to 163 ppb) were prepared by mixing the reverse osmosis water and a dialysis stock solution using a dialysate preparation device (made by NIKKISO CO., LTD., product name: BHI, DRT32) and a dialysate delivery system (made by NIKKISO CO., LTD., product name: DAB-40E). The dialysates thus prepared were delivered to a dialysis machine (made by NIKKISO CO., LTD., product name: DCS-27 or DCS-100NX).

The dialysis stock solution used was a dialysis stock solution for artificial kidneys (made by Fuso Pharmaceutical Industries, Ltd., product name: Kindaly AF-4P).

The concentrations of various electrolytes in the produced dialysates were 140 mEq/L for Na⁺, 2.0 mEq/L for K⁺, 2.75 mEq/L for Ca²⁺, 1.0 mEq/L for Mg²⁺, 112.25 mEq/L for Cl⁻, 8 mEq/L for CH₃COO⁻, 27.5 mEq/L for HCO₃⁻, and 125 mq/dL for glucose (C₆H₁₂O₆). The produced dialysates had a pH in the range of 7.2 to 7.4 and an osmotic pressure ratio in the range of 0.95 to 1.00.

### Evaluation of Feeling of Fatigue

First, a survey was conducted among chronic hemodialysis patients (95 patients, average dialysis duration of 10.6 years) about whether they had a feeling of fatigue on the day of dialysis and the following day. The patients were divided into the following three groups (Groups A to C) in terms of the feeling of fatigue.
Group A: had a feeling of fatigue only on the day of dialysis (40% of 95 people; 38 people)
Group B: had a feeling of fatigue on both the day of dialysis and the following day (11.6% of 95 people; 11 people)
Group C: had no feeling of fatigue (48.4% of 95 people; 46 people)

Next, a hemodialysis (electrolyzed water dialysis) test was performed on each patient on normal hemodialysis (that is, normal dialysis using a dialysate produced in the same manner except that the electrolysis process was not performed using the electrolysis module 7 in FIG. 1) in Groups A to C for eight weeks using the produced dialysates containing electrolyzed hydrogen water. Each patient's feeling of fatigue (severity of the feeling of fatigue) was evaluated before changing to the electrolyzed water dialysis (that is, when the patient was on normal hemodialysis) and two weeks, four weeks, and eight weeks after the start of the electrolyzed water dialysis.

More specifically, each patient in Groups A to C was asked to evaluate the “severity of the feeling of fatigue” using a Visual Analogue Scale (VAS), with 0 indicating no feeling of fatigue and 10 indicating the worst feeling of fatigue possible, regarding the severity of the feeling of fatigue on the day after the dialysis. The scores thus obtained were used as scores for the severity of the feeling of fatigue.

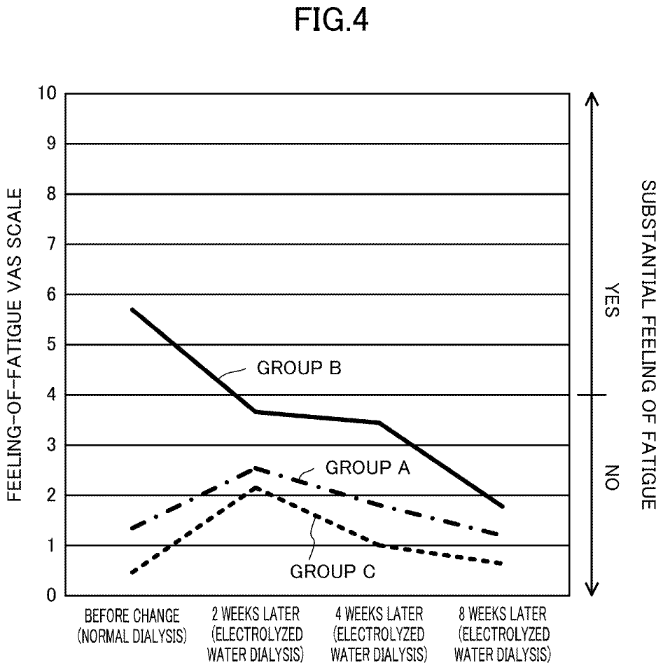

The VAS scores of 4 and higher were evaluated as “had a substantial feeling of fatigue.” The results are shown in FIG. 4.

As shown in FIG. 4, for the patients in Group B who had a feeling of fatigue on both the day of dialysis and the following day, the VAS score decreased to less than 4 two weeks after the start of the electrolyzed water dialysis. This shows that the substantial feeling of fatigue had disappeared. The results also show that the feeling of fatigue decreased to a level similar to the levels of the patients in other groups (Group A and Group C) (i.e., VAS score of less than 2) eight weeks after the start of the electrolyzed water dialysis.

That is, the subjective feeling of fatigue (i.e., the persistent feeling of fatigue from hemodialysis) of the patients with severe fatigue who had a feeling of fatigue not only on the day of dialysis but also on the following day (i.e., the patients in Group B) began to decrease rapidly after the start of the electrolyzed water dialysis with the dialysate of the present invention. The results show that the subjective feeling of fatigue was dramatically reduced in a short period of time (i.e., eight weeks) after the start of the electrolyzed water dialysis with the dialysate of the present invention.

It can be seen from the above that the dialysate containing electrolyzed hydrogen water according to the present invention has an activity of reducing a persistent feeling of fatigue from hemodialysis.

As described above, the present invention is particularly useful for dialysates for hemodialysis.

What is claimed is:

1. A dialysate for hemodialysis containing electrolyzed hydrogen water, having a dissolved hydrogen concentration of 100 ppb to 200 ppb, and having an activity of reducing a feeling of fatigue.

2. The dialysate of claim 1, wherein
the feeling of fatigue is a persistent feeling of fatigue from hemodialysis.
